(19) United States
(12) Patent Application Publication
(10) Pub. No.: US 2016/0213307 A1
(43) Pub. Date: Jul. 28, 2016

(54) Method and apparatus for providing home sleep study chain of custody

(71) Applicants: Burke Lawrence Mays, Franklin, TN (US); Todd Maurice Eiken, Lindstrom, MN (US)

(72) Inventors: Burke Lawrence Mays, Franklin, TN (US); Todd Maurice Eiken, Lindstrom, MN (US)

(21) Appl. No.: 14/999,016

(22) Filed: Mar. 19, 2016

Related U.S. Application Data
(60) Provisional application No. 62/177,642, filed on Mar. 20, 2015.

(51) Int. Cl. A61B 5/00; B65D 51/24; B65D 43/16

(52) U.S. Cl. CPC A61B 5/4806; A61B 5/0077; B65D 43/16; B65D 51/24

(57) ABSTRACT

The present invention is a device and method for assuring “chain of custody” (CoC) in connection with a home sleep study (HST). An HST CoC device according to the invention includes a camera and a circuit board operatively connected to the camera. During the HST the circuit board signals the camera to periodically take images of the patient, to optimally assure CoC. The images are preferably still shots, but can alternatively be relatively short video clips. Further, the images are preferably taken randomly to discourage the patient from attempting to game the system and defeat CoC safeguards. Another embodiment includes a shipping container for a HST device, wherein a CoC device is operatively connected to the shipping container, whereby the CoC device is automatically returned to the lab along with the HST device following the HST.

This claims priority to Provisional Application 62/177,642, filed Mar. 20, 2015.

### FIELD OF INVENTION

This invention relates to home sleep study (HST) methods and apparatus, and in particular “chain of custody” (CoC) systems associated with same.

### BACKGROUND

Sleep studies are tests that measure how well a person sleeps and how a person's body responds to sleep problems. These tests can help a physician determine whether a person has a sleep disorder and, if so, how severe it is. The results of a sleep study also help a physician plan an effective treatment.

Sleep studies are important because untreated sleep disorders can raise the risk for heart disease, high blood pressure, stroke and other co-morbid medical conditions. Sleep disorders also have been linked to an increased risk of workplace injury and the severity and number of truck and auto accidents. It is estimated that one in four men and one in nine women in the US suffer from some type of sleep disorder. Studies estimate that untreated Obstructive Sleep Apnea (OSA), a primary sleep disorder, alone can cost the economy anywhere from $65-165 billion annually.

Sleep studies can be conducted in various ways. A formal, costly technique, called a polysomnogram (PSG), is usually done at a sleep center by a trained technologist. Electrodes are placed on a patient's scalp, face, chest, limbs, and a finger. While sleeping, the electrodes record brain activity, eye movements, muscle activity, respiration/airflow, heart rate/rhythm, blood pressure, and blood oxygenation level. Elastic belts placed around the patient's chest and abdomen measure chest movements and the strength and duration of inhaled and exhaled breaths. Wires attached to the electrodes transmit the data to a computer for analysis. A PSG is an expensive, time-consuming test.

Sleep studies can also be done using a Home Sleep Test (HST) that incorporates fewer physiological parameters and is normally conducted in a person's home, unattended by a trained technologist. An HST is a simplified version of a PSG that focuses on nocturnal breathing. HSTs are only used to diagnose or rule out OSA. Because the emphasis is on breathing, it's possible to use fewer sensors. Patients wear a small monitor by attaching it to a band that wraps around the chest. The monitor records patient breathing via a nasal cannula, respiration, oximetry and pulse. It derives and analyzes a patient's apnea/hypopnea index, flow limitation and snoring in order to create a report with color-keyed risk indicators for clinicians. HST has been shown to provide cost-effective screening for drowsy drivers, high-risk employees (such as truck drivers and night-shift workers), and chronic disease patients.

While HSTs are highly effective for identifying OSA, they are susceptible to CoC issues. The sleep study industry uses CoC to refer to incontrovertible proof that physiological medical data collected during a medical test actually belongs to a particular patient. In the case of HST, this issue has become one of the benchmark measurements for quality HST programs.

Professional drivers have a very high incidence of OSA. An estimated 80% of drivers are sleepy when operating tractor-trailers as a result of OSA induced sleep deprivation. The Congress and the Federal Motor Carrier Safety Administration are moving inexorably toward mandating driver testing for OSA and, depending on the diagnosis, initiation of treatment. Even now, drivers who are identified as at risk for OSA during an annual Department of Transportation mandated bi-annual physical examination must undergo treatment if tested positive for OSA in order to keep their operator's license. Many drivers are understandably resistant to the restraints of treatment and long-term compliance. CoC is especially important when testing of professional drivers given the incentive and opportunity to falsify HST studies.

There are a few CoC solutions commonly employed in the sleep industry, most of which require “face-to-face” equipment and CoC verification. The “gold standard” CoC for HSTs involves a face-to-face meeting between the patient and the technologist administering the test. The technologist places a wrist band on the patient with a sensor wire imbedded in the band material. Once the band is sealed, the only way to remove it is to cut it away. This provides some degree of assurance the person wearing the sensor is the intended patient. This face-to-face gold standard CoC approach adds time, cost, and inconvenience. Additionally, typical HST systems do not employ electroencephalography, used to determine if the patient is asleep. A wrist strap would not prevent the patient from staying awake all night to avoid recording apnea events or sleep disruption. The result is a normal test, no treatment, and a patient who continues to put him/herself and the general public in danger by operating heavy equipment while sleepy.

The present invention addresses the CoC gold standard issue with regard to HSTs without the need for a face-to-face meeting. It provides a simple, effective, and reasonable level CoC assurance, while avoiding undue complexity, inconvenience, and costs.

### SUMMARY OF THE INVENTION

One embodiment of the present invention is an HST CoC assembly including a shipping container for a HST device and a CoC device operatively connected to the shipping container, whereby the CoC device is automatically returned to the lab along with the HST device following the HST. The shipping container can include a base and a lid hingedly connected to the base; and wherein the CoC device is firmly affixed to the lid, whereby when the lid is opened the CoC device may be aimed at a patient.

Another embodiment of the present invention is an HST CoC device including a camera and a circuit board operatively connected to the camera; wherein during an HST the circuit board signals the camera to periodically take images of an HST patient during the HST, to assure CoC. The images are preferably still shots, but can alternatively be relatively short video clips. Further, the images are preferably taken randomly to discourage the patient from attempting to game the system and defeat the CoC provided by the system. This free-standing embodiment of the invention can include a stand on the back panel of the case, whereby the CoC device can be easily “aimed” at the patient. LEDs can be included on the CoC device front panel for illuminating the patient during the recording process.

A third embodiment of the invention is a method of providing CoC in connection with an HST for a patient, including providing the patient with a CoC device having a camera and a circuit board to automatically activate the camera; providing an HST device to the patient; instructing the patient to arrange the CoC device so the camera is aimed at the patient during the HST; instructing the patient to commence the HST; and periodically recording an image of the patient during the HST through activation of the camera by the circuit board, thereby assuring CoC of the HST. This method can further include providing a shipping container for the HST and CoC devices, wherein the CoC device is affixed to the shipping container and is automatically returned to the lab with the HST device upon conclusion of the HST.

Another embodiment of the present invention is an HST CoC device including a case; a camera contained within the case; a circuit board contained within the case; and means for the patient to activate the camera, wherein the camera is activated (i) by the patient to record a statement prior to the HST; and (ii) by the circuit board to periodically record an image of the patient during the course of the HST, whereby HST CoC of the patient is assured. There can be a record button on the case to enable the patient to manually activate the camera. Also, the images recorded automatically by means of the circuit board activating the camera can be still shots or short video clips. Finally, the shots/clips can be taken randomly, to make it difficult for the patient to anticipate when they might be taken, thus better assuring CoC for the entire HST.

Preferred embodiments of the present invention are described with reference to the drawings described below.

### BRIEF DESCRIPTION OF THE DRAWINGS

FIG. 1 shows an enlarged front elevational view of a preferred CoC device;

FIG. 2a shows a back elevational view of the CoC device of FIG. 1;

FIG. 2b shows a side elevational view of the CoC device of FIG. 1;

FIG. 2c shows a perspective view of the CoC device of FIG. 1;

FIG. 3a shows a side elevational view of an HST assembly including a typical shipping container for HST devices and the CoC device of FIG. 1;

FIG. 3b shows a front elevational view of the HST assembly of FIG. 3a;

FIG. 3c shows a perspective view of the HST assembly of FIG. 3a, further including an HST device; and

FIG. 4 shows a functional block diagram of a preferred circuit for the CoC device of FIG. 1.

### DETAILED DESCRIPTION OF THE INVENTION

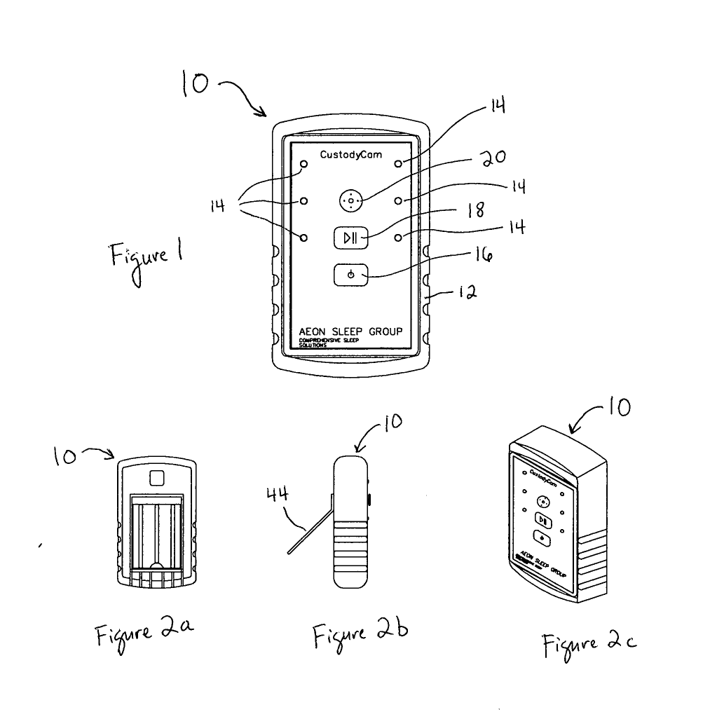

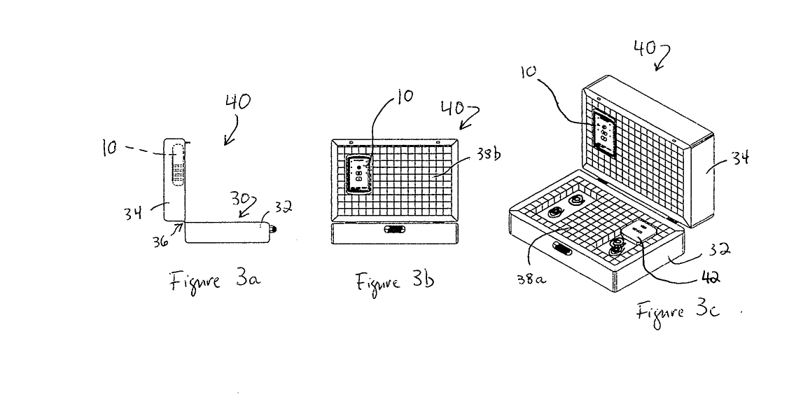

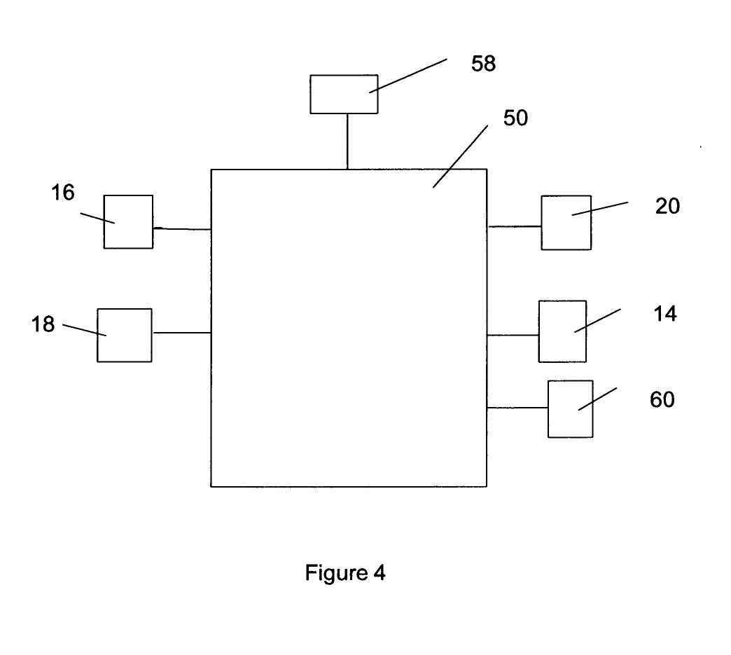

A preferred embodiment of the invention is shown and described with reference to the attached Drawings, wherein like reference numerals designate like parts and assemblies throughout the several views. FIG. 1 shows a preferred CoC device 10 according to the present invention. CoC device 10 can include a rectangular plastic case 12 containing two vertical arrays of LED lights 14 located on the front panel of case 12 toward the outer side edges thereof; a power button 16 toward the bottom of the front panel; a centrally-located record/pause button 18; and a video camera 20 with a wide-angle lens also mounted on the front panel of the case, toward the top center thereof. Camera 20 includes a built-in microphone (not shown), and there is a Micro USB port 60 (shown in FIG. 4 only) mounted on the side of the case 12.

With reference to FIG. 4, the CoC device 10 includes a circuit board 50 attached to which are a rechargeable battery 58, the camera 20, LEDs 14, the Micro USB port 60, power button 16 and record/pause button 18; none of which are shown in detail but all of which would be well understood by those skilled in the art of designing and constructing portable electronic devices. It would further be understood that the circuit board 50 could carry a programmable microcontroller, digital memory, and a timing device or functionality (the purpose of which will be discussed below).

All of the above components (e.g., the battery, digital memory, circuit board, and all the components within case 12) are currently available for sale, and are well known to those skilled in the art. For example, the CoC circuit board discussed above could be a slightly modified version of a circuit board primarily designed for security systems, incorporating video acquisition, audio acquisition, time-and-date stamp, and power buttons. Also, the various components of the CoC device can be arranged differently and still fall within the spirit of this invention. For example, the power button could be located on the side or back panel of the case. Similarly, at least some of the identified components—e.g., the controller, digital memory and timer—can all be incorporated into the circuit board as hardware, firmware or software, or some combination thereof.

A preferred CoC system would also include software for a lab computer (not shown) that would interface with the CoC device 10, before the CoC device is sent to the patient and after the CoC device is returned by the patient to the lab. Such software could create a simple windows user interface for initializing the CoC device's memory (pre-test) with the patient name; providing date and time sync to the CoC device; creating a data file name based on the patient's name and date of acquisition; and downloading the data file (post-test) from the CoC device 10 to the lab computer's memory.

Camera 20 could be a simple digital camera of the type incorporated into many cell phones. As noted above, it would preferably have a wide-angle lens, the reason for which will be made apparent below. While camera 20 could be as simple as described above, it could also have infrared capability; if so, LEDs 14 could be eliminated. Further, camera 20 could take periodic still shots or short video clips, preferably randomly. To set up such still shots or clips, the patient would be instructed to position the CoC device so the camera lens is “aimed” at the patient's bed, following the initial CoC text recitation (described below). Such still shots/clips may further enhance the effectiveness of the CoC device, by periodically recording a digital snapshot/clip of the patient at various times during the test, thus further verifying that the person being tested is indeed the patient. Random shots/clips would be preferred, so the patient could not predict when they would be taken.

CoC device 10 could use a memory card to store its data, a battery life indicator, Bluetooth capability, and finger print capability. In fact, versions of the CoC device of the present invention could be integrated into an HST device (Bluetooth, wireless, and/or hard connection) to “marry” video, stills, and audio to the physiological data file; and even include the ability to superimpose video, still shots, and audio data over the physiological data wave forms.

One preferred embodiment of the present invention would couple the CoC device 10 with a typical shipping container 30 used to ship HST devices back and forth between testing service providers or labs and patients. Container 30 includes a base 32 and a lid 34 coupled together by means of a hinge 36. The base 32 and lid 34 are lined with foam 38. Specifically, base 32 is lined with base foam 38a, and lid 34 is lined with lid foam 38b.

With further reference to FIG. 3, a combination shipping container/CoC device 40, or what could be termed an “HST assembly,” includes a CoC device 10 mounted to the inside of lid 34. The foam 38b is cut away from the lid, and the CoC device 10 is attached, preferably epoxied, to the underside of the lid. Similarly, the base foam 38a could be shaped to receive a standard HST device 42. Patients are instructed prior to beginning the HST to place the container 30 on their knees while sitting in a chair; lift lid 34 so the CoC device is “aimed” right at the patient; press the power button 16 to power up the CoC device 10; press the record/ pause button 18 to start the video recording; recite prescribed text (described below); and then re-press the pause/record button 18 to stop the video recording. Following that initiation procedure, the patient simply aims the CoC device at his or her bed, and the CoC device periodically captures images of the patient during the HST. The period of time between image captures could be adjustable, perhaps anywhere from a few minutes to an hour or more, depending on a variety of factors, e.g., the level of concern that the patient may try to game the system. Upon completing the HST, the shipping container 30 is used to ship the HST device 42 (and the CoC device 10 by virtue of the fact it is firmly attached to the underside of container lid 34) back to the testing provider, or the lab.

It should be noted that the HST assembly 40 could be arranged as shown in the Drawings, where the CoC device 10 is securely attached to the shipping container lid; but assembly 40 could also be arranged and configured such that the CoC device 10 is connected to another portion of the shipping container, or, for that matter, integrated into the shipping container. All that is required for this particular embodiment of the invention is that the CoC device 10 be operatively coupled to the shipping container, so that when the shipping container is returned to the lab (for example), that in effect automatically and seamlessly returns the CoC device 10 to the lab. Automatically returning the CoC device increases the integrity of the chain of custody evidence or proof established by the CoC device; but also increases the likelihood that the CoC device won't be stolen, harmed, or forgotten, by the patient or someone else.

Further with regard to the process, at the same time that an HST device 42 is plugged into a lab computer and initialized with the patient name, and date and time synced with the computer, the CoC device is also initialized with the same information (using a simple software program written for the device). Preferred HST and CoC devices both contain date/time stamp capability, and they are synced up prior to sending out. Batteries are charged for both devices and the container is shipped to the patient. The patient applies the HST electrodes while preparing for bed, then just prior to beginning the HST test sits on the side of the bed and places the shipping container 30 on his or her knees, and lifts the lid to “aim” the CoC camera 20 and built-in microphone at himself or herself. The patient then pushes the power and record buttons 16 and 18. The 6 LEDs 14 illuminate, and the video and audio acquisition begins. The patient recites prescribed text, e.g.:

“My name is John Doe, the date is ______ and the current time is ______. I have applied all the electrodes for my home sleep test and am now beginning the test.”

Upon reading the prescribed text, the patient presses the record/pause button 18 which stops the recording and turns off LEDs 14. If the patient never pushes button 18 to stop recording the initial reading of the prescribed text, the recording will time out after a preset period of time, for example 3 minutes.

Following the recording of the prescribed text, the patient simply positions the device 40 with the lid 34 open, such the camera 20 points in the general direction of the patient's bed. The wide-angle lens makes it unnecessary for the aiming process to be overly precise. Then, the circuit board (typically a controller on the circuit board) periodically, automatically activates the camera 20 to capture an image of the patient, preferably a still shot but alternatively a short video clip. After a preset period of time, possibly 6-7 hours, the circuit board will cease automatic, periodic activation of the camera.

The next morning, the patient returns the HST device 42 and related cords and electrodes to the shipping container 30 and sends it back to the testing service provider. Importantly, the CoC device 10 is “automatically” returned as well, by virtue of the fact that it is securely attached to the underside of container lid 34.

Upon return, the testing service provider downloads the HST data to the lab computer for analysis, and also downloads the small, time and date stamped video/audio file from CoC device 10 via the Micro USB port (not shown) to the same computer. The CoC filename will include the patient's name and acquisition date. The CoC file is then placed within the patient's HST data folder, for further reference as needed.

It will be appreciated that the present invention provides advantages not previously available in the HST industry. For example, one advantage associated with the preferred embodiment where the CoC device is attached to, or integrated with, the HST device shipping container, is that the CoC device will be reliably returned with the HST device. It should also be appreciated that the above-described embodiments are only illustrative of the application of the principles of the present invention. Numerous modifications and alternative arrangements may be devised by those skilled in the art without departing from the spirit and scope of the present invention; and the appended claims are intended to cover such modifications and arrangements. For example, the CoC device needn't be firmly affixed to the lid of the shipping container; that is, the CoC device could be easily removable from the shipping container, so the user would simply need to prop up the CoC device using stand 44 prior to commencing the test, with the camera aimed toward the patient so as to capture the initial statement and also the periodic shots/clips designed to assure CoC.

What is claimed is:

1. An HST CoC assembly comprising a shipping container for a HST device and a CoC device operatively connected to the shipping container, whereby the CoC device is automatically returned to the lab along with the HST device following the HST.

2. The HST CoC assembly of claim 1, wherein the shipping container comprises a base and a lid hingedly connected to the base; and wherein the CoC device is firmly affixed to the lid, whereby when the lid is opened the CoC device may be aimed at a patient.

3. A HST CoC device comprising a camera and a circuit board operatively connected to the camera; wherein during an HST the circuit board signals the camera to periodically take images of an HST patient during the HST, to assure CoC.

4. The HST CoC device of claim 3, wherein the images are still shots.

5. The HST CoC device of claim 3, wherein the images are taken randomly.

6. The CoC device of claim 3, further comprising a stand on the back panel of the case.

7. The CoC device of claim 3, further comprising an LED for illuminating the user during the recording process.

8. A method of providing CoC in connection with an HST for a patient, comprising:
a. Providing the patient with a CoC device having a camera and a circuit board to automatically activate the camera;
b. Providing an HST device to the patient;
c. Instructing the patient to arrange the CoC device so the camera is aimed at the patient during the HST;
d. Instructing the patient to commence the HST; and
e. Periodically recording an image of the patient during the HST through activation of the camera by the circuit board, thereby assuring CoC of the HST.

9. The method of claim 8, further comprising providing a shipping container for the HST and CoC devices, wherein the CoC device is affixed to the shipping container and is automatically returned to the lab with the HST device upon conclusion of the HST.
